(19) United States
(12) Patent Application Publication
(10) Pub. No.: US 2009/0062676 A1
(43) Pub. Date: Mar. 5, 2009

(54) PHASE AND STATE DEPENDENT EEG AND BRAIN IMAGING

(75) Inventors: Stepan Kruglikov, Geneva (CH); Steven Schiff, Chevy Chase, MD (US)

Correspondence Address: MILLEN, WHITE, ZELANO & BRANIGAN, P.C., 2200 CLARENDON BLVD., SUITE 1400, ARLINGTON, VA, 22201, US

(73) Assignee: GEORGE MASON INTELLECTUAL PROPERTY, Fairfax, VA (US)

(21) Appl. No.: 10/555,794

(22) PCT Filed: May 6, 2004
(86) PCT No.: PCT/US2004/014067

§ 371 Date: Nov. 19, 2008

Related U.S. Application Data
(60) Provisional application No. 60/468,087, filed on May 6, 2003.

(51) Int. Cl. A61B 5/0476

(52) U.S. Cl. 600/544

(57) ABSTRACT

The present invention provides methods and devices for performing electroencephalographic (EEG) phase dependent brain imaging using evoked and event related potentials (EP, ERP) or other forms of brain imaging including functional magnetic resonance imaging (fMRI) and magnetic encephalography (MEG). The methods and devices can be used for a variety of purposes, including for the study of normal and pathological cognitive function, and for the diagnosis and prognosis of neurological and sleep disorders.

This application claims the benefit of U.S. Provisional Application Ser. No. 60/468,087, filed May 6, 2003.

### DESCRIPTION OF THE FIGURES

FIG. 1 (A-B) shows a method of determining brain phase using threshold and time delay.

FIG. 2 shows a method of determining brain state using pattern-based similarity searching.

FIG. 3 (A-D). Three schemas illustrating the interaction between stimuli, EEG, and the neural correlates of evoked potentials: I, traditional view of stimuli interacting with neural circuitry independently of EEG, II, phase resetting view of stimuli interacting with EEG, and III, schema suggested by the results of this study, whereby neural correlates of stimulation and ongoing EEG modulate each other in a time dependent fashion. B. Schematic of experimental apparatus. C. Example of 10 raw unstimulated phase triggered trials from an individual subject. D. Example of experimental protocol for a subject.

FIG. 4 (A-C). Example of single epoch of data from one subject. Upper panel shows raw EEG signal. Stimuli, S, indicated as vertical solid lines, were delivered at 0 and 500 ms. Horizontal dashed line indicates threshold, T, for extracting phase at π/−π. Second panel shows Hilbert transform retrospectively derived phase. B. Averages of the difference between phase triggered and unstimulated phase control trials from one subject at 0, 25, 50, and 75 ms delay. Latencies for P30 and P50 peaks indicated with vertical dashed lines, and despite changes in amplitude with phase, the latencies are constant. Also shown are averages from regular stimulation intervals, and sampled irregular stimulation intervals. C. Origin of phase triggered evoked potentials from averages of phase triggered (solid line) and unstimulated phase control trials (dashed line) at 0 ms delay for this subject. P30 and P50 evoked potentials derive from fluctuations in EEG amplitude along similar initial phases of the EEG cycle. Insets show progressive expansion in time scale, with raw difference below, and at bottom, filtered (10-50 Hz) difference customarily employed to extract P30 and P50.

FIG. 5. Grand average results from 20 subjects. Solid lines represent averages at 0 ms (red), 25 ms (green), 50 ms (blue), and 75 ms (black). Dashed lines indicate bootstrapped confidence intervals (p=0.025). Insets demonstrate increases and decreases in amplitude of P30 at 0 ms and 50 ms respectively, and a comparable decrease and increase in P50 amplitude at 0 ms and 50 ms respectively. Inset waveforms aligned as detailed in Methods. No significant effects of first stimulus phase are seen in P30 and P50 measured at second stimulus 500 ms later.

FIG. 6 (A-B). Pooled phase histograms from 20 subjects, indicating the distribution (counts) of Hilbert transform derived phases at the onset of stimuli for 0, 25, 50, and 75 ms delay phase triggered stimuli, as well as regular and sampled irregular stimuli. For unstimulated (left column) and first tone stimuli (middle column), there is a progressively less restricted set of phases as one progresses from 0 through 25 and 50 ms delays, and a nearly uniform distribution of phases at 75 ms delay. Rayleigh statistics parameter R and Bonferroni corrected p value, p′, are shown for each distribution (significant results with p′<0.0001 are denoted by an asterisk). Regular and sampled stimuli phases for first tone, and all phase distributions for second tone stimuli are uniformly distributed. B. Averaged evoked potential peak-to-peak amplitudes to first (left column) and second (right column) tones. Mean amplitudes indicated by heavy horizontal bars. The significant (asterisks, *) increases in P30 and P50 amplitudes for first tone stimuli at 0 and 50 ms delays respectively, and the significant decreases in P30 and P50 amplitudes at 50 and 0 ms delays respectively, are quantified in the text.

### DESCRIPTION OF THE INVENTION

The present invention provides methods and devices for determining brain phase, and for performing phase and state dependent imaging using modalities such as electroencephalograms (EEG), magnetic encephalography (MEG), and functional resonance imaging (fMRI). The methods and devices can be used for a variety of purposes, including for diagnostic purposes, to determine brain state (e.g., using evoked potential as a measure); to produce an indication of how anesthetized or how awake a patient is; for the study and diagnosis of neurological (e.g., seizure disorders, tumors, head injuries, degenerative diseases and brain death) and sleep disorders; and for correlating EEG or other types of brain-derived signals with a behavior or psychiatric or neurological condition, mood, mental performance, attention, and vigilance. The present invention also provides methods and devices for determining the effect of agents on the brain, including psychotropic and other pharmacologically-active agents, where phase or state dependent brain imaging can be used to assess neurological effects. In addition, phase and state dependent stimulation methods and devices can be used in combination with diagnostic, feedback, behavior modification, and therapeutic methods that utilize EEG or other modalities that capture signals from the brain.

“Brain phase” as used herein relates to characteristics of the oscillatory electrical activity of the brain recorded using electrodes (scalp, intracranial, extracellular). These oscillations represent the field potentials of the neurons that comprise the brain. When the oscillatory electrical activity of the brain is recorded using electrodes placed on the scalp of the subject, it is referred to as an electroencephalogram (“EEG”). In EEG recordings, the field potentials appear as electrical transient events. EEG activity can be broken down into distinct frequency bands: (1) Beta activity, 13 Hz-32 Hz; (2) Alpha activity, 8 Hz-13 Hz; (3) Theta activity, 4 Hz-7 Hz; (4) Delta activity, <4 Hz, and (5) Gamma, >32 Hz. Each of these represents a synchronized and oscillatory activity that can be analyzed in accordance with the methods of the present invention. Beta activity is normally present when the eyes are open or closed. Alpha activity is also a normal activity observed in waking adults. It is predominantly recorded from electrodes placed in the back of the head. It is fairly symmetrical and generally has an amplitude of about 40 μV to 100 μV. The amplitude of alpha activity is most commonly seen when the eyes are closed, and disappears or is reduced in amplitude when the eyes are open. Theta activity is both a normal and abnormal activity, depending on the age and state of the patient. In adults, it is normal when it occurs in a drowsy subject, but its appearance can also indicate brain dysfunction in a subject who is alert and awake. Delta activity is only normal in an adult subject when in a moderate to deep sleep. At any other time, it is considered to indicate brain dysfunction. Gamma activity is intimately related to sensory perception and cognitive events.

The term “phase” refers to labeling the periodicity of the waveforms (from −π to +π, or from 0 to 2π, etc.) of the particular brain activity (electrical, magnetic, metabolic, etc.) that is being measured. Determining “brain phase” therefore refers to determining the position within a period, or one complete cycle, of the periodic waveform, as measured at a point in time. It is the same as labeling the position of the hand of a clock in terms of minutes of an hour, where the hour represents the period or complete cycle in question.

“State” refers to the condition of the brain, which may or may not be reflected in a periodic phase. The pattern based similarity method allows for determination of the brain state whether it is periodic and phasic, or without a well-defined periodic cycle and phase.

Any method of recording and detecting the phase or state of brain activity can be utilized. Therefore, although the disclosure may refer specifically to EEG, the methods are applicable to other means for recording brain phase or state.

### Determining Brain Phase

Brain activity can be assessed by any measurable characteristic or signal that can be recorded from it, including electrical, magnetic, and metabolic signals. The activity can be characterized as phasic, where a periodicity can be identified. The present invention provides methods of determining the phase of the brain with respect to the measured activity.

The present invention relates to methods for determining the EEG phase of a subject, comprising one or more of the following steps, e.g., recording an EEG from a subject's brain, and calculating a voltage amplitude threshold value from the EEG, whereby said threshold value corresponds to a value of phase of said EEG.

EEG recording can be performed conventionally. For example, multiple electrodes can be placed on the surface of the scalp at specific positions. The set of locations is called a montage. The International 10/20 System is an example of a widely used montage. A montage can comprise monopolar electrodes, where each electrode records electrical activity with reference to a distant site, such as the ear lobe. Bipolar montages can also be utilized, where the electrodes are interconnected and reference each other. Various systems are available commercially for displaying and recording data, e.g., storing data in a storage means. Single channel EEG is when only one electrode is used (recorded or analyzed). Multiple electrode recordings produce multiple channel EEG.

For general references on EEG recording, see, e.g., Davidson et al., In: Cacioppo et al., editors, Handbook of Psychophysiology. Cambridge: Cambridge University Press, 2000:27-56; American Electroencephalographic Society Guidelines for Standard Electrode Position Nomenclature, Journal of Clinical Neurophysiology, 1991, 8(2):200-202; Niedermeyer E. & Lopes Da Silva F H. (Editors). Electroencephalography: Basic Principles, Clinical Applications, and Related Fields. Williams & Wilkins, 1998; Nunez P L. Electric Fields of the Brain: The Neurophysics of EEG, Oxford Univ. Press, 1998; Cooper et al., EEG Technology, 2nd ed., Butterworths, London, 1969.

The present invention provides methods for determining a value of phase for an EEG comprising calculating a “voltage amplitude threshold value.” (Voltage can be used when the signal is electrical, but other signal measurements can also be used, e.g., current, magnetic, temperature, light intensity, etc) In this embodiment, a baseline EEG recording is collected for a subject over a period of time (“threshold-determining period”). To obtain the voltage amplitude threshold value, the most negative or positive of the amplitudes are identified, and then sorted by their numerical value. The uppermost or lowermost set of values selected as a threshold is defined as the “voltage amplitude threshold value.” This value can be determined routinely, e.g., by creating a histogram of all values and selecting the uppermost or lowermost limit.

Each time a signal amplitude crosses this threshold, i.e. is below or above the value, depending on whether the uppermost or lowermost set of values was chosen, the EEG phase is considered to be at the same point in phase. Any range of values can be defined as the threshold value, e.g., the most negative (or positive) about 10%, about 5%, about 4%, about 2%, about 1%, about 0.5%, etc., and any value in between. As shown in the examples below, using a threshold that corresponded to deep troughs in the EEG, signals were identified with nearly identical phase. This can be confirmed retrospectively using Hilbert or wavelet transformations.

The period over which the threshold is determined is arbitrary, e.g., over 10,000 seconds, over 1,000 seconds, over 100 seconds, over 10 seconds, etc., but generally is of sufficient length that a sufficient number of complete cycles or period of the EEG are recorded.

The method is equally applicable to other measurement methods from which the phase of cyclic activity of the brain, or non-cyclic brain state, can be determined, such as magnetic encephalography (MEG), functional magnetic resonance imaging (fMRI), etc. For example, when MEG is utilized, the changes in magnetic field that are recorded over time can be used to calculate brain phase analogously to how the EEG signal amplitudes are utilized. When fMRI is used as the imaging modality, the phase can be determined in any given region of the brain over time, where the metabolic changes can be correlated with the time component. When the EEG phase is calculated using voltage amplitude threshold, and determined just prior to the EEG data collection session, it can be referred to as “real-time” to indicate that calculation is being performed instantaneously or coincidentally with the experimental recording. Phase can also be calculated using standard techniques, such as Hilbert or wavelet transformation. See, e.g., Barlow JS. The Electroencephalogram: Its Patterns and Origins, Cambridge, Mass.: MIT Press, 1993, Chapt. 29, p. 356-363; Hahn SL, Hilbert Transforms in Signal Processing, Boston: Artech House, 1996; Stearns DS, and David R A. Signal Processing Algorithms in Matlab, Upper Saddle River, N.J.: Prentice Hall/Simon and Schuster, 1996. These are acausal and retrospective, requiring information about the signal in the future from the time point at which the phase is calculated, and therefore are not readily applicable to real-time recording. Bendat and Piersol. Random data: analysis and measurement produces, Ed 3, pp 518-543. New York: Wiley, 2000.

The methods of determining phase described herein have wide applicability to the study of brain activity, especially in combination with EEG, MEG, or fMRI recording. A common way to assess electrophysiological function in individuals is with an electroencephalogram (EEG). The response of the brain to a stimulus can be used to assess and diagnose the condition of a subject. The response to a stimulus which is observed in an EEG is generally referred to as an event related or evoked potential (“EP”). There is no limitation as to the type of stimulus utilized, and these stimuli include, but are not limited to, auditory, visual, electrical, mechanical (touch, pressure, vibration, or joint movement stimulus), olfactory, and taste stimuli.

EPs are widely used to assess neurological function. For example, EPs produced by an auditory stimulus can be utilized to evaluate the auditory function of an infant; sensory EP produced by low current delivered to the skin is used during spine surgery to monitor the integrity of the spinal cord; visual evoked potentials are used to assess various abnormalities of the visual system. Since the brain's excitability and sensitivity to stimulation is dependent upon its baseline phasic activity, the actual phase at the time the stimulus is delivered may effect and influence the resulting EP. The present invention provides methods and devices for addressing this concern.

When eliciting an EP, the response to the stimulus is superimposed on the ongoing phasic activity of the brain, and therefore the observed evoked potential actually represents a composite of the actual response and the normal background electrical activity. The present invention provides a method of dissociating the EP from the brain's ongoing electrical or other measurable activity.

### Phase Dependent Triggering

The present invention also provides methods for using the brain phase to trigger the presentation of a stimulus to a subject. This permits the researcher to take into account the state of the brain when analyzing its response to the stimulus, and to administer a plurality of stimuli to the brain, each delivered at the same brain phase.

The present invention provides methods of detecting and recording an evoked potential of a stimulus, comprising one or more of the following steps, in any effective order, e.g., (1) recording an EEG in the presence and absence of a stimulus, and optionally wherein the stimulus is triggered at a defined position in the EEG phase, (2) aligning, in phase, (a) a segment of the EEG recorded in the absence of the stimulus with (b) a segment of the EEG recorded in the presence of the stimulus, and (3) subtracting (a) the EEG record in the absence of the stimulus from (b) the EEG record in the presence of the stimulus, whereby the net difference between (a) and (b) is the evoked potential of said stimulus, and wherein the records are aligned in phase when subtracted.

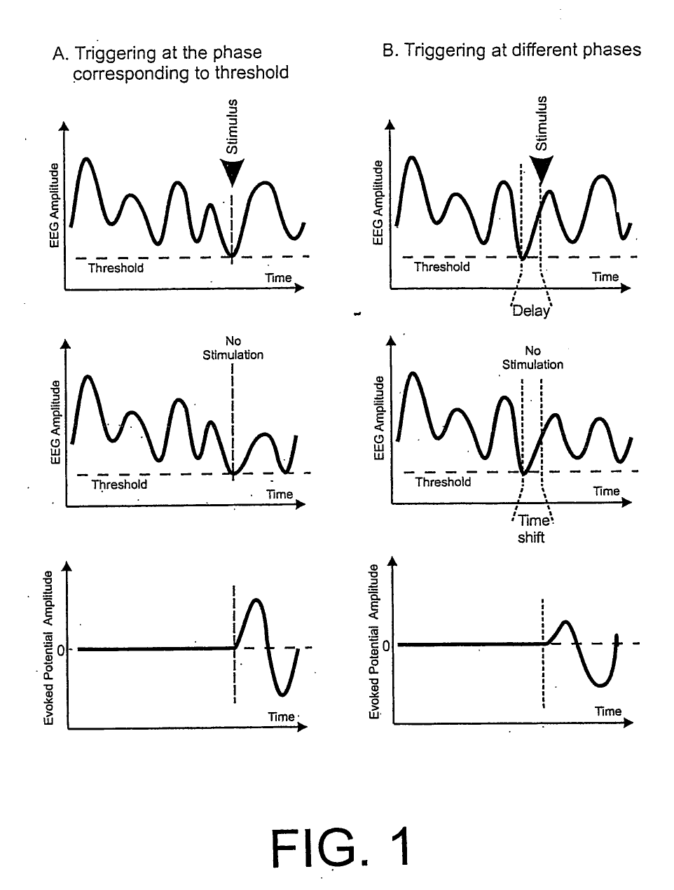

A first part of the method involves determining the phase of the EEG, and then delivering the stimulus at defined phase period. Methods of determining phase have been described above, particularly where amplitude threshold value is utilized in real-time. Once a threshold value is determined, it can be used to trigger the presentation (delivery) of a stimulus to a subject in such manner that it occurs at precisely the same phase period (FIG. 1A). The stimulus can be delivered immediately when the threshold value is reached, or at any desired time delay from it (FIG. 1B).

Once the phase of the EEG waveform has been determined, the present invention provides methods of removing the ongoing activity from the record to produce a record of the evoked potential that is “free” of the phase background that can also be referred to as the phase artifact. The latter manifests itself as spurious features (troughs or peaks) at stimulus onset and beyond.

The methods generally involve aligning, in phase, a segment of the EEG recorded in the absence of the stimulus (stimulus-absent) with an EEG segment recorded in the presence of the stimulus (stimulus-present), and then removing the stimulus-absent record from the stimulus-present record. The aligning process can be implemented by selecting an EEG segment in the absence of a stimulus, and then calculating its phase. The length of the record can be of any size that is useful for performing the mathematical analysis, e.g., about 1000 msec, 100 msec, 10 msec, etc., such that the record has at least one complete cycle. This stimulus-absent record is then phase-matched to a stimulus-present record (FIG. 1).

The phase-matching can be done routinely, e.g., where each point in the first record is assigned a phase value and then matched to the corresponding phase value in the second record. Once the two sets of phase-matched values have been created, any desired method can be used to remove the stimulus-absent values from the stimulus-present values. For example, the stimulus-absent values can be mathematically subtracted from the stimulus-present values to create a “processed” evoked potential record that is free of the phase artifact (FIG. 1). Others means for removing the stimulus-absent record from the stimulus-present record can be utilized, e.g., where subtraction is combined with statistical analysis and average weighting.

A single recording can be utilized for the analysis, where one stimulus-absent record and one stimulus-present record are processed to remove the phase artifact. But, also, multiple records of each type can be used, where averaging and other statistical methods are used to process the information. For example, EEG epochs can be collected from a single or multiple recording sessions, averaged and then processed as described above. In addition, the averaging can occur after the processing step. Statistical methods can also be used to eliminate trials from the analysis when they do not meet some criteria. The EEG can be filtered to establish phase within a more narrow frequency range.

The stimulus can be triggered at a defined position in the EEG phase. For example, a real-time method of determining phase can be utilized to assign phase to an ongoing waveform. When a preselected phase point is reached (between −π and +π), the stimulus is delivered to the subject. This can be repeated continuously through a single recording session, providing a way of comparing EPs that are observed during a single or multiple recording sessions. Thus, when more than one trial is utilized for analysis, the various EPs that are observed during the session can be phase-matched and averaged together.

Phase dependent stimulus triggering can be employed concurrently with non-phase dependent imaging. That is, an image can be acquired independently of the phase or state triggered stimulus. For continuously acquired data, one can trigger the stimulus while acquiring the images continuously. Similarly, one can trigger both the acquisition of the image and stimulus in a phase or state dependent manner. Our methodology applies to both the timing of delivery of a stimulus, and the timing of the imaging.

### Pattern-Based Similarity Methods

The present invention also provides methods of establishing brain state using pattern-based similarity searching. The pattern based similarity method allows for determination of brain state, whether it is periodic and phasic, or whether it is without a well defined periodic cycle and phase.

This embodiment can comprise one or more of the following steps in any effective order, e.g., (a) recording an EEG in the presence and absence of a stimulus, wherein said stimulus is delivered without regard to EEG phase, (b) comparing the just pre-stimulus EEG record to the unstimulated EEG record to find the closest pattern match between the two records, and (c) subtracting the closest pattern match of the subsequent unstimulated EEG record from the time-matched just post-stimulus EEG record, whereby the net difference is the evoked potential of said stimulus.

In this method, a query sequence is identified from an EEG recording, and this query sequence is used as a probe against other parts of the EEG record to identify a region of local similarity.

In step (a), an EEG is recorded from a subject in the presence and absence of a stimulus, wherein said stimulus is delivered without regard to EEG phase. Thus, an EEG record is created which comprises both pre-stimulus brain activity, and the evoked potential elicited by the stimulus. As before, any type of stimulus can be utilized. Once the EEG record has been created, it can be processed to eliminate the state dependent (or phase) artifact from the EP. This processing can be performed using pattern-based similarity searching.

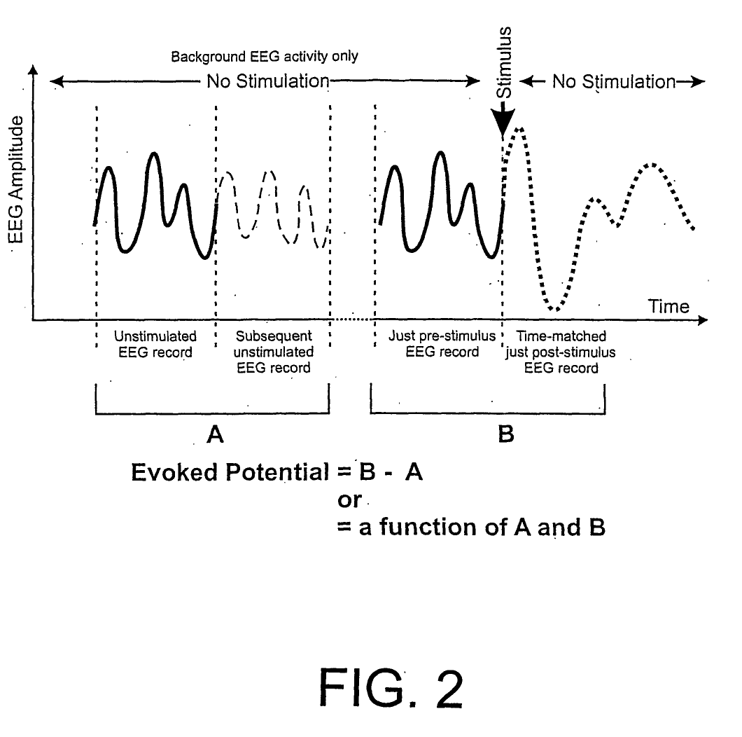

For example, the method can comprise (b) comparing the just pre-stimulus EEG record to the unstimulated EEG record to find the closest pattern match between the two records. FIG. 2 illustrates these features of the EEG record. The “just pre-stimulus EEG record” can be the segment of the complete EEG record just prior to delivery of the stimulus. This segment is utilized as a query or probe to search other segments of the EEG record. As shown in FIG. 2, the just pre-stimulus EEG record can be substantially contiguous with the segment during which the stimulus is delivered. This choice may be preferred because the just pre-stimulus record is utilized to define the state of the brain at the time of stimulus delivery, and it therefore is logical that it be from substantially the same frame of reference as when the stimulus is actually delivered.

However, in some circumstances it may be useful to use an archived pre-stimulus EEG record for analysis purposes. For example, EEG collections can be made of EEG sessions from various activity states (such as sleeping, under anesthesia, meditating, after administration of a drug or other pharmacological agent, in a defined emotional mood, in a defined physical state, in a defined arousal state, etc.)

The just pre-stimulus EEG record can then compared to the unstimulated EEG record to find the closest pattern match between them. As shown in FIG. 2, the unstimulated EEG record can be the segment of the EEG record from the same recording session, but prior to stimulus delivery. The closest match represents the segment of the EEG record that shows the most or highest similarity to the query pattern. Similarity searching can be performed routinely. For example, features of the pre-stimulus record can be extracted, and then used to search the unstimulated portion of the EEG record. Features, include voltage amplitude, duration, direction (i.e., positive or negative), shape, frequency, spike rate (e.g., number per time unit), power spectral density, etc. The EEG record can be defined by filtering, fast wavelet, Fourier transforms, and other well-known transformations. Statistical correlation, coherence, matched template analysis, matching pursuit analysis, mutual information, and other means can be used to quantify similarity. For pattern (similarity) matching both query pattern and unstimulated EEG records can be downsampled to accelerate the process of similarity search.

The segment of the unstimulated record having the closest match to the just pre-stimulus record defines the start of the EEG record which is used to remove the state or phase artifact from the evoked potential. The part of the record immediately following the match is referred to as the “subsequent unstimulated EEG record.” In step (c), the closest pattern match of the subsequent unstimulated EEG record is removed from the time-matched just post-stimulus EEG record, whereby the net difference is the evoked potential of said stimulus. The removal of the subsequent unstimulated record from the time-matched post-stimulus record can be done by simple subtraction, but other methods can be used, as well. For example, the records can be processed by an algorithm, and then resultant values can be subtracted.

The phrase “time-matched just post-stimulus EEG record” indicates the segment of the EEG record after the stimulus has been delivered which corresponds to the same time-frame as the unstimulated EEG record with respect to the closest match. For example, if the duration of the just pre-stimulus record is 100 msec, and it immediately precedes a 100 msec period at the start of which the stimulus was delivered, then the subsequent unstimulated EEG record would be aligned with the just post-stimulus EEG record 100 msec after the start of the match. This point is shown in FIG. 2 as the arrow marked “Stimulus.” In other words, the EEG segment from the subsequent unstimulated record is aligned temporally with the post-stimulated record so when the records are subtracted, the appropriate corresponding time points are subtracted from each other.

The pattern based similarity method is equally applicable to other measurement methods from which the state of activity of the brain, whether cyclic or non-cyclic, can be determined, such as from magnetic encephalography (MEG), functional magnetic resonance imaging (fMRI), etc.

### Phase Dependent Imaging

The methods of determining brain phase described herein can be used for any purpose, including for diagnostic purposes and in brain imaging. For example, the present invention can be used to produce an image of the brain at a desired phase of the EEG cycle. The EEG signal can be employed as a time base for triggering the imaging signal or to gate its acquisition, and/or can be used to trigger stimulation at a consistent phase of the EEG. Images can be produced using any modality, including, but not limited to, MRI, fMRI, MEG, CT, PET, FDG-PET, SPECT, EEG, ultrasound, etc. Using EEG phase to trigger or gate the imaging modality, permits the collection of images at the same position in the EEG, compensating for non-uniform changes in the brain cycle.

Phase dependent imaging and analysis can be carried analogously to cardiac gating as described, e.g., in U.S. Pat. Nos. 6,539,074, 6,535,754, 6,526,117, 6,516,210, 6,510,337, 6,421,552, 6,393,091, 6,370,217, 6,329,819, 6,310,479, 6,278,765, 6,275,560, 6,234,968, 6,154,516, 6,078,175, 6,070,097, 5,997,883, 5,871,013, 5,830,143, 5,458,126, 5,352,979, 5,251,628, 4,991,587, 4,881,032, 4,716,368, 4,547,892, 3,954,098, Am. J. Roentgenol., 180:505-512, 2003, Am. J Neuroradiology, 23: 225-230, 2002, etc., which are hereby incorporated by reference in their entirety. It can also be carried analogously to respiratory gated imaging, e.g., as described in U.S. Pat. Nos. 4,694,837, 5,485,835, and 6,704,593, which are hereby incorporated by reference in their entirety.

In one embodiment of the present invention, the present invention relates to methods and devices for imaging the brain of a subject, comprising collecting a plurality of images of a brain at defined phases of an EEG. In typical brain imaging, the images are collected over a series of time, irrespective of the brain phase. When brain images are then compared from one time point to another, they represent the brain in a different and random periodicity, and therefore observed differences may reflect phasing artifact, rather than true dissimilarities. The present invention proves a way of performing imaging that takes into account the brain phase.

In this method, a plurality of images (e.g., at least two) can be collected from the same defined phase (i.e., at any phase value from −π to +π). These images can be collected continuously, where a series of temporally continuous images are utilized for analysis, or where images are sampled at various times. The plurality of images at the same phase value can then be utilized to provide an image of the brain at a single EEG phase. The brain phase can be determined by any method, but is preferably determined by the amplitude threshold value as described above.

The imaging can be performed using any available technique, including, but not limited to electrode arrays, magnetic encephalography (MEG), magnetic resonance imaging (MRI), functional magnetic resonance imaging (fMRI), positron emission tomography (PET), fluoro-deoxyglucose positron emission tomography (FDG-PET), or single photon emission computed tomography (SPECT). A device to be used with the phase imaging can incorporate an EEG apparatus with the imaging device, where the EEG is utilized to trigger the capture of an image and/or the delivery of a sensory stimulus.

The phase imaging of the present invention can also be used in combination with any methods or processes of collecting information about the brain. For example, the images can be collected in the presence or absence of a stimulus, where the images are compared at the same brain phase. This method is analogous to the evoked potential method described, but where the images are analyzed to obtain an image of the brain that correlates with the evoked potential.

Methods and devices for recording an electroencephalogram during magnetic resonance imaging (MRI) are described in, e.g., U.S. Pat. No. 5,445,162 (Ives). For simultaneous recording of fMRI and EEG data, see also, e.g., Garreffa et al., Magn. Reson. Imaging. 2003 December; 21(10):1175-89; Salek-Haddadi et al., Mag. Reson. Imaging. 2003 December; 21(10):1159-66; Al-Asmi et al., Epilepsia. 2003 October; 44(10):1328-39; Benar et al., Clin Neurophysiol. 2003 March; 114(3):569-80. For simultaneous recording of PET and EEG data, see, e.g., Oakes et al., Hum Brain Mapp. 2004 April; 21(4):257-70; Sadato et al., Neuroreport. 1998 Mar. 30; 9(5):893-7. For simultaneous recording of SPECT and EEG data, see, e.g., Spanaki et al., Epilepsia. 1999; March; 40(3):267-74; Bye et al., Clin Exp Neurol. 1993; 30:117-26.

The topic headings set forth above are meant as guidance where certain information can be found in the application, but are not intended to be the only source in the application where information on such topic can be found.

Without further elaboration, it is believed that one skilled in the art can, using the preceding description, utilize the present invention to its fullest extent. The following preferred specific embodiments are, therefore, to be construed as merely illustrative, and not limiting of the remainder of the disclosure in any way whatsoever. The entire disclosure of all applications, patents and publications, cited above and in the figures are hereby incorporated by reference in their entirety, including U.S. Provisional Application Ser. No. 60/468,087, filed May 6, 2003.

### EXAMPLES

### Methods

### Experimental Procedures

Twenty normal healthy subjects (10 males), 20 to 30 years old, were recruited from the campus of George Mason University, under a protocol approved by the university's Institutional Review Board. Subjects took no prescription medications and had no hearing problems. They were paid $50 US following testing.

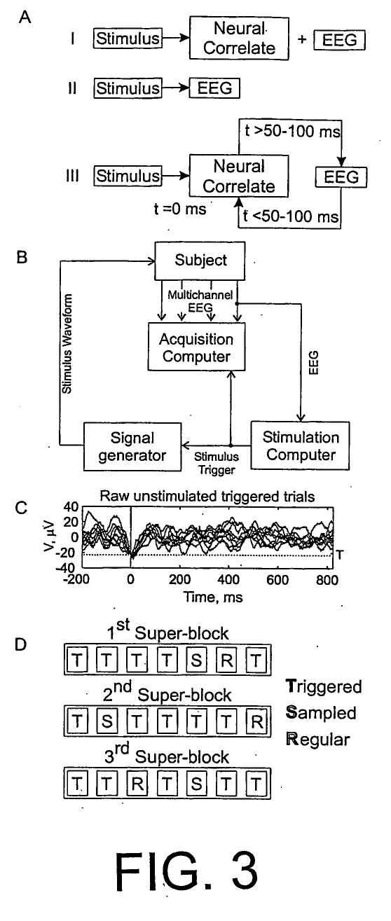

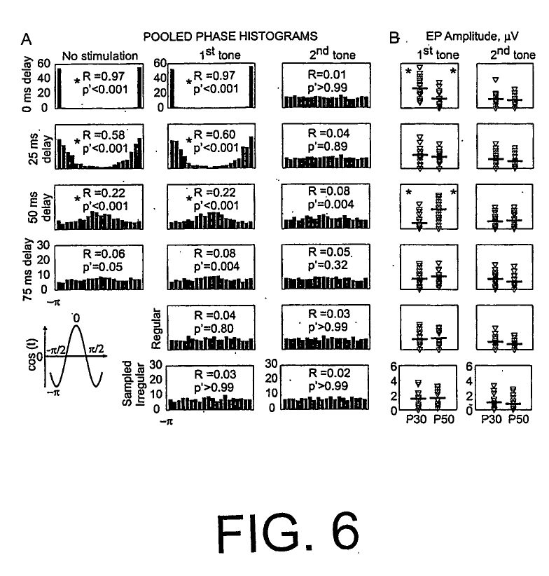

EEG/EP equipment was modified to present auditory stimuli timed to the phase of the ongoing EEG (FIG. 3B). Employing phase in real time is complicated by the fact that the methods generally used to directly calculate phase, such as Hilbert or wavelet transforms, are acausal, i.e. require information about the signal in the future from the time point at which the phase is calculated, and cannot be applied in real time (Bendat and Pierson, 2000). Therefore, for each subject, voltages during a 100 s baseline recording at the beginning of the experiment (relaxed eyes closed) were used to set a threshold to identify the most negative 1% of amplitudes. Employing a threshold that corresponded to deep troughs in the EEG was found to identify signals with nearly identical phases (calculated retrospectively). Ten unstimulated triggered individual trials are shown in FIG. 3C, visually illustrating how phase is aligned at 0 ms. By selecting various delays (0, 25, 50, 75 ms) following this threshold, we could trigger stimuli in a phase dependent manner. In FIG. 6A, we illustrate the statistical significance of such phase dependence.

Four types of trials were presented. The first two types employed auditory stimuli timed to EEG phase (with 4 different delays), matched with unstimulated phase control trials where phase was determined through threshold but no stimuli were presented. In order to match unstimulated phase control trials to phase triggered stimuli, we shifted control trials backwards in time by 25, 50, or 75 ms as required. We could thus control for the waveform morphology associated with EEG phase by subtracting this from the trials with auditory stimuli triggered from EEG phase.

This control has not previously been used. Without such a control, phase selective averaging of EEG creates the ‘prestimulus phase bias’ (Makeig et al., 2002), which has been observed in previous work (Jansen and Brandt, 1991). Use of a phase control allowed us to extract the neural correlates of the EP from the phase dependent waveform.

For farther comparison, we presented two types of stimuli without regard to ongoing EEG phase: one set at regular intervals of 3.5 s with random jitter of ±0.3 s, and another set using interstimulus intervals resampled from the phase triggered trials. EEG dependent triggering increases the variability of interstimulus intervals (Rahn and Basar, 1993), and it is known that longer and irregular intervals can increase the amplitude of EPs (Makeig 1990). To take this into account, we presented stimuli with intervals sampled from the previous phase triggered trials, presenting them in blocks without regard to phase.

Because of the clinical interest of paired pulse P50 suppression for the study of sensory gating, we delivered a second auditory stimulus at fixed 500 ms delay (Freedman et al., 1987). Our working hypothesis was that the response to the second tone might also be affected by the phase at which the first tone was presented.

An example of our block design is shown in FIG. 3D. Three super-blocks containing equal numbers of each type of stimuli were presented. Each super-block contained seven blocks with 50 stimulus presentations in each block. The order of the blocks were randomized within each super block, with the restriction that the sampled stimulus interval block could not occur before at least 2 phase triggered blocks were performed. Fifteen random sequences of 50 phase triggered trials were created, each sequence consisting of 10 presentations of each of the 5 types of phase triggered stimuli. Five of these sequences were presented in random order within each super block, so that a total of 750 phase triggered stimuli were given (150 of each type) in the entire experiment. To ensure uniformity, the same 15 random sequences (with the same stimulus type order) were used for each subject, but in randomized block orders. The sampled interval blocks were constructed by randomly selecting phase triggered intervals from the pool of all previous triggered intervals (0, 25, 50, 75 ms, and unstimulated phase control trials).

Subjects were relaxed with eyes closed during stimuli, and were asked to open their eyes and stay alert between blocks. Each block lasted from 3 to 5 min, and the entire experimental protocol required on average 90 min to complete. Seventeen of twenty ( 17/20) subjects completed the entire protocol (150 trials of each type), and the remaining three ( 3/20) subjects completed two of three super-blocks (100 trials of each type).

Stimuli were produced by a signal generator, which generated two 20 ms 1000 Hz tones, 500 ms apart, at 65 dB sound pressure level above hearing threshold at 1000 Hz, delivered to the subject via insert earphones (Etymotic Research model 3A). An EEG cap (Neuroscan QuickCap) with Ag—AgCl electrodes was applied according to the 10-20 system, and EEG passed through a biopotential isolation unit (Grass IMEB-2NUM25), analog filtered (0.3-100 Hz, −3 dB), amplified with gain 10,000 (Grass model 12), digitized at 1 kHz across 12 bits (Digidata 1200A, Axon Instruments), and recorded on an acquisition computer. The width and roll-off of the analog bandpass filter did not significantly distort phase nor create appreciable phase delay in the region of the dominant EEG frequencies in the alpha band. No further online digital filtering was applied prior to determination of threshold or retrospective analysis of phase. Electrodes were applied using conductive gel, and impedances kept below 5 kΩ, using electrodes F3, Fz, F4, C3, Cz, C4, P3, Pz, P4, bipolar HEOG, bipolar VEOG, and linked ear references. Single channel EEG from electrode CZ, which yields the largest amplitude mid-latency EP responses (Nagamoto et al., 1991), was simultaneously digitized at 2500 Hz across 16 bits (PCI-MIO-16XE10, National Instruments) for a separate stimulation computer, which controlled the signal generator.

Phase was calculated in broad band from Hilbert transformation (HT) of the signal. HT is defined as

$$h ( t ) = \frac{1}{π} {lim}_{ɛ \to 0} \{ {\int }_{- ∞}^{t - ɛ} \frac{x ( τ )}{t - τ} d τ + {\int }_{t + ɛ}^{+ ∞} \frac{x ( τ )}{t - τ} d τ \} ,$$

where x(t) is the original signal (Bendat and Piersol, 2000). The Gabor analytic signal Z(t) is defined as Z(t)=x(t)+ih(t)=a(t)e^{iφ(t)}, and the phase of the signal can be obtained as

$$ϕ ( t ) = {tan}^{- 1} \frac{h ( t )}{x ( t )} .$$

Numerically the Gabor analytic signal was obtained by calculating FFT of the signal, multiplying the result by

$$s ( f ) = \{ \begin{matrix}2 , f > 0 \\ 1 , f = 0 \\ 0 , f < 0\end{matrix} ,$$

where f denotes frequency, and then performing inverse FFT of the product (Bendat and Piersol, 2000). Note that the four-quadrant inverse tangent was used, and therefore the phase was within the interval [−π; π].

Phase was determined in broad band (0.3-100 Hz), and this study performed without narrow band filtering of signals. On the one hand, the signals of interest, P30 and P50, lay outside of, for instance, the alpha (8-13 Hz) band often selected for such phase studies. Furthermore, broad band phase assignments through Hilbert transformation is a powerful means to assign phase to biological signals, and may avoid possible artifacts introduced during phase assignment on narrow band filtered signals (Netoff and Schiff, 2002). Although there was prominent alpha band power in many of our traces, the method we present is applicable regardless of the dominant (e.g., alpha, theta, etc.) cortical cycling present. Our approach is to apply the least amount of selective filtering of these signals as possible.

Data Analysis

Epochs were extracted from 200 ms before until 823 ms after stimulation onset (1024 discrete voltages). Artifact rejection was applied, rejecting epochs if the amplitude in the bipolar horizontal (HEOG) or vertical electrooculogram (VEOG) exceeded 75 μV in absolute value.

Standard procedures were used to identify P30 and P50. P30 peak-to-peak amplitude was found as the peak at the maximum between 25-45 ms (Kisley et al., 2001) following stimulus onset, and its amplitude was measured with respect to the preceding negativity. P50 was similarly determined in the window 45-85 ms (Nagamoto et al., 1991; Jin, et al., 1997). Prior to extraction of P30 and P50 amplitudes, we applied a 10-50 Hz bandpass digital 5^{th} order Butterworth filter with 3 dB rolloff, applied with zero phase shift filtering technique (forward and reverse) using Matlab function ‘filtfilt’ (Mathworks). Such a bandpass filter reduces the effect of N100 (Jerger et al., 1992) and is commonly used to extract the positive evoked potential peaks near 30 (P30) and 50 (P50) ms.

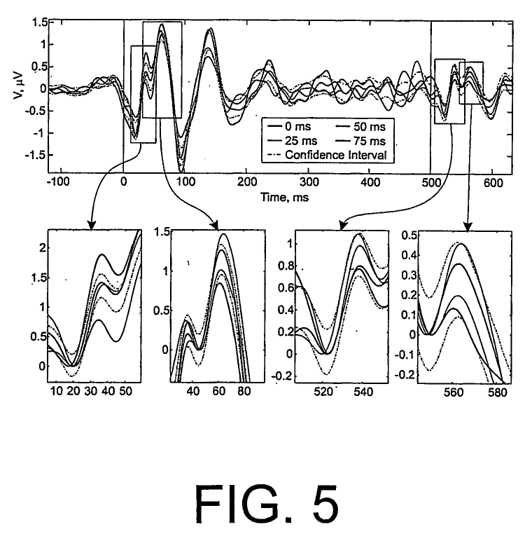

The significance of the effect of delay on EP components was also evaluated by performing a bootstrap analysis on the pool of phase triggered trials from all subjects. This bootstrap tested the null hypothesis that there was no effect of delay on EP amplitude, and was constructed as follows. First, out of a possible 3000 epochs at each delay, we retained 2850 after subject task incompletion (loss of 150 epochs). After artifact rejection (approximately 10% rejected), there remained 2640, 2663, 2668, and 2649 phase triggered epochs of each type. For each subject, the average of unstimulated phase controls, appropriately shifted to match delays, was subtracted from each phase triggered stimulated trial. The pool thus consisted of all controlled phase triggered trials from all subjects at each of the 4 phase delays. We chose the average, 2655, as the number of trials to form resampled (bootstrap) averages. From the pool of all phase triggered trials (n=10620) we randomly, without regard to the phase, selected 2655 trials, and calculated a bootstrap average. The procedure of resampling and averaging was repeated 1000 times. The 1000 new averages formed a distribution from which the probability of a given voltage at each time point could be determined. We selected confidence limits corresponding to the probability that a given voltage value would have p≦0.025 chance of exceeding the value. To evaluate individual P30 and P50 peak-to-peak amplitudes, the preceding negativities before P30 and P50 were realigned to zero voltage by subtracting the average value of preceding negativity (N20 and N40) from each individual epoch, and the confidence intervals recalculated in order to determine the significance of peak to peak excursions. This process was repeated for each evoked potential amplitude shown in the insets of FIG. 5.

### Results

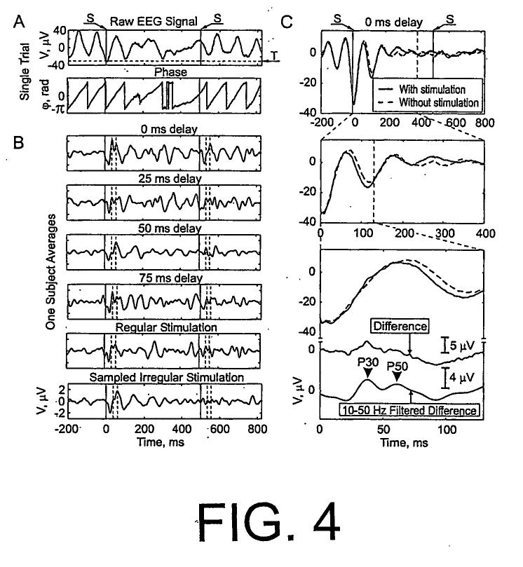

An example of the relationship of calculated phase to raw EEG is shown in FIG. 4A. Phase goes through zero at the positive peaks in the EEG signal, and abruptly shifts from π to −π at the troughs. By triggering off of the large troughs phase could be precisely (at 0 ms delay) determined from amplitude. The differences between averages of the phase triggered and unstimulated control trials are shown in the upper panels of FIG. 4B for delays of 0, 25, 50, and 75 ms following threshold. In the lower panels of FIG. 4B are shown the average responses to regular and sampled irregular stimuli. Note that P30 and P50 average latencies are not affected by these different stimulus conditions (dashed vertical lines in FIG. 4B). This absence of effect on latency was confirmed for all subjects by repeated measures analysis of variance: P30 (df=3, F=1.72, p=0.19), P50 (df=3, F=1.54, p=0.22).

To illustrate the difference between EEG phase with and without stimuli, we plot for one subject the average responses to stimulated and unstimulated phase control trials for 0 ms delay in FIG. 4C. At the expanded time scale in the middle panels of FIG. 2C, one sees that the P30 and P50 are due to amplitude fluctuations in the signals with little shift in the phase of the dominant EEG frequency. This is in clear contrast to the apparent phase shift in the trough that accounts for a significant fraction of the N100. Note in the lower panel of FIG. 4C, the bandpass filtered trace (10-50 Hz) used to extract P30 and P50 amplitudes (Jerger et al., 1992).

Grand average results for all 20 subjects are shown in FIG. 5. We resampled epochs without regard to phase, in order build a bootstrapped confidence statistic that tested the null hypothesis that phase was irrelevant to evoked potential amplitude (see Methods). As can be seen in the insets of FIG. 5 recalculated to evaluate each P30 and P50, the response to the first stimulus was strongly affected by the estimated phase of the EEG, while the P30 and P50 amplitudes in response to the stimulus 500 ms later had no such effect. Whereas the first P30 amplitude was significantly increased at 0 ms delay and decreased significantly at 50 ms delay, the amplitude of first P50 was significantly decreased at 0 ms delay, and significantly increased at 50 ms delay. At 25 and 75 ms delays, no significant effect on peak amplitudes were observed.

The interstimulus intervals (ISIs) for sampled triggered stimulation were significantly longer (mean 5.1 s; median 4.4 s; σ=2.3) than regular stimulation (mean 3.5 s; median 3.5 s; σ=0.17) by t-test (p<0.001, t=−36, df=5388). Nevertheless, we observed no significant difference between EP amplitudes for regular and sampled triggered stimulation: P30 (t=−0.39, p=0.70), P50 (t=−0.34, p=0.73).

We examined the relationship between phase delay and, retrospectively, the actual calculated phases of the EEG at the time of stimulus onset in FIG. 6. The relationship between 0 delay and phase was extremely precise to/As delay was increased, the distribution of phases became spread out, and was far more uniform at 50 ms delay than at 0 ms (left and middle column FIG. 6A). We employed Rayleigh statistics as a test for randomness against a unimodal distribution (Fisher, 1993) for all phase distributions shown in FIG. 6A. This statistic sums vectors of the phases (‘phasors’) to obtain an amplitude, R, which varies from 0 (uniform distribution of phases) to 1 (identical) phases. In each panel of FIG. 6A we show the R values, along with Bonferroni corrected (16 comparisons) significance (p′). The Rayleigh statistics confirmed the significant association of phase with delay at 0, 25, and 50 ms, for both unstimulated and stimulated trials.

We further tested whether there was any difference between the stimulated and unstimulated distributions at the different delays. Circular rank based W statistics (Fisher, 1993) which test whether two circular distributions are identical, failed to reveal any differences between phase distributions from trials with stimulation and phase control trials (W_{0 ms}=0.22, p=0.90; W_{25 ms}=0.55, p=0.76; W_{50 ms}=0.26, p=0.88; W_{75 ms}=1.06, p=0.59).

There were very significant (opposite) effects on P30 (0 vs. 50 ms delay) and P50 (0 vs. 50 ms delay) peak to peak amplitudes, shown in the left column of FIG. 4B, by parametric paired t-tests (p<0.001, t=8.3 for P30_{0 ms} vs P30_{50 ms}, t=−4.7 for P50_{0 ms} vs P50_{50 ms}), nonparametric Wilcoxin signed rank test (p<0.001, z=3.9 for P30₀ ms vs P30_{50 ms}, z−3.5 for P50_{0 ms} vs P50_{50 ms}), and phase has a significant effect by repeated measures ANOVA with delay as a factor (df=3, 57, F=23, p<0.001, for P30; df=3, 57, F=10, p<0.001, for P50), as well as the Friedman test (Glantz and Glantz, 2001), a nonparametric analog to this ANOVA (df=3, χ²=31, p<0.001 for P30; df=3, χ²=17, p<0.001 for P50).

For normal subjects the P50 sensory gating ratio, defined as a fraction of the response to the second tone amplitude to the first, should be less than one (Freedman et al., 1987). We found that the P50 ratio was significantly less than unity (one sample t-test) for all conditions, including regular and sampled stimulation, except for 0 ms delay (t=0.1, =0.93). Unexpectedly, the P30 ratio, generally expected to be unsuppressed (Kisley et al., 2001), was found significantly less than unity for 0 and 25 ms delays (t=−6.86, p<0.001; t=−3.17, p=0.005). The repeated measures (using delays at 0, 25, 50, 75 ms as a factor) analysis of variance on the P30 and P50 peak-to-peak amplitudes for the second tone did not show a significant effect of delay: P30 (df=3, F=0.86, p=0.46), P50 (df=3, F=0.15, p=0.92). Thus the differences in sensory gating ratio were due to the effects shown above of phase triggered stimuli on the first P30 and P50.

### REFERENCES

Arieli A, Sterkin A, Grinvald A, Aertsen A (1996) Dynamics of ongoing activity: explanation of the large variability in evoked cortical responses. Science 273:1868-1871.
Bechtereva N P, Zontov V V (1962) The relationship between certain forms of potentials and variations in brain excitability (based on EEG, recorded during photic stimuli triggered by rhythmic brain potentials). Electroencephalogr Clin Neurophysiol 14:320-330.
Bendat J S, Piersol A G (2000) Random Data, pp 51-543. New York: J. Wiley & Sons.
Bishop G (1933) Cyclic changes in excitability of the optic pathway of the rabbit. Am J Physiol 103: 213-224.
Brandt M E (1997) Visual and auditory evoked phase resetting of the alpha EEG. Int J Psychophysiol 26:285-298.
Callaway E, Yeager C L (1960) Relationship between reaction time and electroencephalographic alpha phase. Science 132:1765-1766.
Dustman R E, Beck E C (1965) Phase of alpha brain waves, reaction time and visually evoked potentials. Electroencephalogr Clin Neurophysiol 18:433-440.
Freedman R, Adler L E, Gerhardt G A, Waldo M, Baker N, Rose G M, Drebing C, Nagamoto H, Bickford-Wimer P, Franks R (1987) Neurobiological studies of sensory gating in schizophrenia. Schizophr Bull 13:669-78.
Glantz S S, Glantz S A (2001) Primer of Biostatistics, p 376. New York: McGraw Hill.
Fisher NI (1993) Statistical analysis of circular data, p 70. New York: Cambridge University Press.
Jansen B H, Brandt M E (1991) The effect of the phase of prestimulus alpha activity on the averaged visual evoked response. Electroencephalog Clin Neurophysiol 80:241-250.
Jansen B H, Agarwal G, Hedge A, Boutros N N (2003) Phase synchronization of the ongoing EEG and auditory EP generation. Clin Neurophysiol 114: 79-85.
Jerger K, Biggins C, Fein G (1992) P50 suppression is not affected by attentional manipulations. Biol. Psychiatry 31: 365-377.
Jin Y, Potkin S G, Patterson J V, Sandman C A, Hetrick W P, Bunney W E (1997) Effects of P50 temporal variability on sensory gating in schizophrenia, Psych. Res. 70: 71-81.
Kisley M A, Gerstein G L (1999) Trial-to-trial variability and state-dependent modulation of auditory-evoked responses in cortex. J Neurosci 19:10451-10460.
Kisley M A, Olincy A, Freedman R (2001) The effect of state on sensory gating: comparing of waking, REM and non-REM sleep. Clin Neurophysiol 112: 1154-1165.
Lindsley D B (1952) Psychological phenomena and the electroencephalogram. Electroencephalogr Clin Neurophysiol 4:443-456.
Lopes da Silva F (1999) Even-related potentials: methodology and quantification. In: Electroencephalography: basic principles, clinical applications, and related fields (Niedermeyer E and Lopes da Silva F, eds), pp 947-957. Philadelphia: Lippincott Williams & Wilkins.
Makeig S (1990) A dramatic increase in the auditory middle latency response at very slow rates. In: Psychophysiological Brain Research (Brunia C, Gaillard A, Kok A, eds), pp 56-60. Taliburg: Tilburg UP.
Makeig S, Westerfield M, Jung T P, Enghoff S, Townsend J, Courchesne E, Sejnowski (2002) Dynamic brain sources of visual evoked responses. Science 295:690-694.
Nagamoto H T, Adler L E, Waldo M C, Griffith J, Freedman R (1991) Gating of auditory response in schizophrenic and normal controls. Schizophr Res 4: 31-40.
Netoff T I, Schiff S J (2002) Decreased neuronal synchronization during experimental seizures, J Neurosci 22: 7297-7307.
Polich J (1997) On the relationship between EEG and P300: individual differences, aging, and ultradian rhythms. Int J Psychophysiol 26: 299-317.
Rahn E, Basar E (1993) Prestimulus EEG-activity strongly influences the auditory evoked vertex response: a new method for selective averaging. Int J Neurosci 69:207-220.
Remond A, Lesèrve N (1967) Variations in average visual evoked potential as a function of the alpha rhythm phase (“Autostimulation”). Electroencephalogr Clin Neurophysiol Suppl 26: 42-52.
Rice D M, Hagstrom E C (1989) Some evidence in support of relationship between human auditory signal-detection performance and the phase of the alpha cycle. Percept Mot Skills 69:451-457.
Tass P A (1999) Phase Resetting in Medicine and Biology, pp 227-229. Berlin: Springer.
Tsodyks M, Kenet T, Grinvald A, Arieli A (1999) Linking spontaneous activity of single cortical neurons and the underlying functional architecture. Science 286:19431-946.

1. A method for determining the EEG phase of a subject, comprising,
recording an EEG from a subject's brain, and
calculating a voltage amplitude threshold value from the EEG, whereby said threshold value corresponds to a value of phase of said EEG.

2. A method of claim 1, wherein said amplitude threshold is calculated retrospectively.

3. A method of claim 1, wherein said value of phase of said EEG is further calculated by Hilbert or wavelet transformation.

4. A method of claim 1, wherein said amplitude threshold is calculated in real time.

5. A method of claim 1, further comprising,
wherein the value of said EEG phase is assigned at a timed delay from the voltage amplitude threshold value.

6. A method of claim 1, wherein the voltage amplitude threshold value is the most negative 0.5% or less of the distribution of amplitudes measured from said EEG

7. A method of claim 1, wherein the voltage amplitude threshold value is the most negative 1% or less of the distribution of amplitudes measured from said EEG.

8. A method of claim 1, wherein the voltage amplitude threshold value is the most negative 5% or less of the distribution of amplitudes measured from said EEG.

9. A method of claim 1, wherein the voltage amplitude threshold value is the most positive 0.5% or greater of the distribution of amplitudes measured from said EEG.

10. A method of claim 1, wherein the voltage amplitude threshold value is the most positive 1% or greater of the distribution of amplitudes measured from said EEG.

11. A method of claim 1, wherein the voltage amplitude threshold value is the most positive 5% or greater of the distribution of amplitudes measured from said EEG.

12. A method for determining the phase of a subject's brain with respect to a magnetic encephalography (MEG), or functional magnetic resonance (fMRI) signal, comprising:
recording a MEG or fMRI signal from a subject's brain, and
calculating a signal amplitude threshold value from the MEG or fMRI recording, whereby said threshold value corresponds to a value of phase.

13. A method of detecting and recording an evoked potential of a stimulus, comprising,
recording an EEG in the presence and absence of a stimulus, wherein the stimulus is triggered at a defined position in the EEG phase,
aligning, in phase, (a) a segment of the EEG recorded in the absence of the stimulus with (b) a segment of the EEG recorded in the presence of the stimulus, and
subtracting (a) the EEG record in the absence of the stimulus from (b) the EEG record in the presence of the stimulus,
whereby the net difference between (a) and (b) is the evoked potential of said stimulus, and wherein the records are aligned in phase when subtracted.

14. A method of claim 13, wherein said stimulus is an auditory, visual, electrical, magnetic, mechanical, thermal, olfactory, or taste stimulus.

15. A method of claim 14, wherein said mechanical stimulus is a touch, pressure, vibration, or joint movement stimulus.

16. A method of claim 13, wherein said comparison is performed for single or multiple channel EEG.

17-20. (canceled)

21. A method of imaging the brain of a subject, comprising, collecting a plurality of images of a brain at defined phases of an EEG according to claim 1.

22. A method of claim 21, wherein said image is acquired using electrode arrays as EEG, magnetic encephalography (MEG), magnetic resonance imaging, functional magnetic resonance imaging, positron emission tomography, fluoro-deoxyglucose positron emission tomography, single photon emission tomography.

23. A method of claim 21, wherein said image is acquired using optical imaging.

24. A method of claim 23, wherein said optical imaging uses a near infrared imaging, an intrinsic optical signal, or an absorption or fluorescent optical dye.

25-26. (canceled)
